(19) United States
(12) Patent Application Publication
(10) Pub. No.: US 2021/0099645 A1
(43) Pub. Date: Apr. 1, 2021

(54) ENDOSCOPE SYSTEM, ENDOSCOPIC IMAGE GENERATING METHOD, AND NON-TRANSITORY COMPUTER-READABLE RECORDING MEDIUM

(71) Applicant: OLYMPUS CORPORATION, Tokyo (JP)

(72) Inventors: Takanori USHIJIMA, Tama-shi, Tokyo (JP); Susumu AONO, Hachioji-shi, Tokyo (JP)

(73) Assignee: OLYMPUS CORPORATION, Tokyo (JP)

(21) Appl. No.: 17/122,412

(22) Filed: Dec. 15, 2020

Related U.S. Application Data
(63) Continuation of application No. PCT/JP2019/004611, filed on Feb. 8, 2019.

(30) Foreign Application Priority Data
Jul. 3, 2018 (JP) 2018-127059

(51) Int. Cl. H04N 5/232; H04N 5/225; H04N 13/239; H04N 13/332; H04N 7/18; A61B 1/045; A61B 1/00; A61B 1/06; G02B 23/24; G02B 30/34

(52) U.S. Cl. CPC H04N 5/23229; H04N 2005/2255; H04N 5/2253; H04N 13/239; H04N 13/332; H04N 7/181; H04N 5/2256; A61B 1/045; A61B 1/00048; A61B 1/06; A61B 1/00055; G02B 23/2415; G02B 23/2484; G02B 23/2461; G02B 30/34; H04N 5/23299

(57) ABSTRACT

An endoscope system includes an endoscope, an image generating unit, a display unit, a displayed-image controlling unit, and a distance sensing unit. The endoscope includes an image pickup optical system including a first image pickup device and a second image pickup device. The distance sensing unit senses distance information. Based on the distance information, the displayed-image controlling unit controls a first picked-up image so as to change a position of a first output region included in an entire image pickup region of the first image pickup device and in which an image pickup signal for the first picked-up image is outputted, and controls a second picked-up image so as to change a position of a second output region included in an entire image pickup region of the second image pickup device and in which an image pickup signal for the second picked-up image is outputted.

### CROSS REFERENCE TO RELATED APPLICATION

This application is a continuation application of PCT/JP2019/004611 filed on Feb. 8, 2019 and claims benefit of Japanese Application No. 2018-127059 filed in Japan on Jul. 3, 2018, the entire contents of which are incorporated herein by this reference.

### BACKGROUND OF THE INVENTION

### 1. Field of the Invention

The present invention relates to an endoscope system, an endoscopic image generating method, and a non-transitory computer-readable recording medium for composing two acquired images of an object to be perceived as a stereoscopic image.

### 2. Description of the Related Art

In recent years, endoscope devices have been widely used in medical and industrial fields. Endoscope devices used in the medical field include an elongated insertion portion inserted into a body, and have been widely used for observation of organs, therapeutic devices using treatment instruments, surgical operations under endoscopic observation, and the like.

A common endoscope device is an endoscope device that observes a portion to be observed in a planar image. In the case of a planar image, when it is desired to observe minute asperities on a surface of a body cavity wall or the like as a to-be-observed portion or grasp a spatial positional relationship between organs and devices in a body cavity, for example, perspective and three-dimensional appearance cannot be obtained. Thus, in recent years, a three-dimensional endoscope system that enables three-dimensional observation of an object has been developed.

As a method for enabling three-dimensional perception of an object, there is a method in which two images having a parallax are picked up by two image pickup devices provided in the endoscope and the two images are displayed as a 3D image on a 3D monitor, for example. In this method, an observer perceives a stereoscopic image by seeing the 3D image separately with left and right eyes using 3D observation glasses such as polarizing glasses.

Depending on a distance between the object and an objective, the stereoscopic image may be hard to recognize and unnatural feeling and eyestrain may be caused. As a method for resolving difficulty in recognizing the stereoscopic image, there is a method of resolving difficulty in recognizing the entire stereoscopic image by performing predetermined processing on a region that is hard to observe such as an inconsistent region of left and right images, as disclosed in Japanese Patent Application Laid-Open Publication No. 2005-334462, Japanese Patent Application Laid-Open Publication No. 2005-58374, and Japanese Patent Application Laid-Open Publication No. 2010-57619, for example.

### SUMMARY OF THE INVENTION

An endoscope system in an aspect of the present invention includes: an endoscope including a first image pickup device and a second image pickup device each configured to pick up an image of an object in a subject; a monitor configured to display a 3D image as a displayed image; a sensor configured to sense distance information that is information of a distance from an objective surface positioned at a distal end of an insertion portion of the endoscope to a predetermined observation object in the subject; and a processor, wherein the processor is configured to: generate the 3D image based on a first picked-up image picked up by the first image pickup device and a second picked-up image picked up by the second image pickup device; and change the displayed image by performing at least one of control of the endoscope, image processing for generating the 3D image, and control of the monitor, and based on the distance information, the processor controls the first picked-up image so as to change a position of a first output region included in an entire image pickup region of the first image pickup device and in which an image pickup signal for the first picked-up image is outputted, controls the second picked-up image so as to change a position of a second output region included in an entire image pickup region of the second image pickup device and in which an image pickup signal for the second picked-up image is outputted, controls the first and second picked-up images such that an interval between a center of the first output region and a center of the second output region is a first value when the distance is within a predetermined range, and controls the first and second picked-up images such that the interval is a second value different from the first value when the distance is out of the predetermined range.

An endoscopic image generating method in an aspect of the present invention is an endoscopic image generating method for generating a 3D image based on first and second picked-up images respectively picked up by first and second image pickup devices of an endoscope, the endoscopic image generating method including: sensing distance information that is information of a distance from an objective surface positioned at a distal end of an insertion portion of the endoscope to a predetermined observation object in a subject; and based on the distance information, controlling the first picked-up image so as to change a position of a first output region included in an entire image pickup region of the first image pickup device and in which an image pickup signal for the first picked-up image is outputted, controlling the second picked-up image so as to change a position of a second output region included in an entire image pickup region of the second image pickup device and in which an image pickup signal for the second picked-up image is outputted, controlling the first and second picked-up images such that an interval between a center of the first output region and a center of the second output region is a first value when the distance is within a predetermined range, and controlling the first and second picked-up images such that the interval is a second value different from the first value when the distance is out of the predetermined range.

A non-transitory computer-readable recording medium in an aspect of the present invention is a non-transitory computer-readable recording medium storing an endoscopic image processing program to be executed by a computer, wherein the endoscopic image processing program causes an endoscopic image generating system for generating a 3D image based on first and second picked-up images respectively picked up by first and second image pickup devices of an endoscope to perform: sensing distance information that is information of a distance from an objective surface positioned at a distal end of an insertion portion of the endoscope to a predetermined observation object in a subject; and based on the distance information, controlling the first picked-up image so as to change a position of a first output region included in an entire image pickup region of the first image pickup device and in which an image pickup signal for the first picked-up image is outputted, controlling the second picked-up image so as to change a position of a second output region included in an entire image pickup region of the second image pickup device and in which an image pickup signal for the second picked-up image is outputted, controlling the first and second picked-up images such that an interval between a center of the first output region and a center of the second output region is a first value when the distance is within a predetermined range, and controlling the first and second picked-up images such that the interval is a second value different from the first value when the distance is out of the predetermined range.

### BRIEF DESCRIPTION OF THE DRAWINGS

FIG. 1 is an explanatory diagram showing a schematic configuration of an endoscope system according to an embodiment of the present invention;

FIG. 2 is a functional block diagram showing a configuration of the endoscope system according to the embodiment of the present invention;

FIG. 3 is explanatory diagram showing an example of a hardware configuration of a main-body device in the embodiment of the present invention;

FIG. 4 is an explanatory diagram schematically showing a range in which a three-dimensional image of a stereoscopic image can be comfortably observed with regard to a distance between a 3D monitor and an observer;

FIG. 5A is an explanatory diagram schematically showing a range in which a three-dimensional image of the stereoscopic image can be comfortably observed with regard to a distance between an observation object and an objective and showing a distal end portion of an insertion portion and the observation object;

FIG. 5B is an explanatory diagram schematically showing a range in which a three-dimensional image of the stereoscopic image can be comfortably observed with regard to the distance between the observation object and the objective and showing the three-dimensional image of the observation object;

FIG. 6 is an explanatory diagram showing image pickup regions and output regions of image pickup devices in the embodiment of the present invention;

FIG. 7 is an explanatory diagram showing a situation after positions of the output regions are changed from a situation shown in FIG. 6;

FIG. 8A is an explanatory diagram showing a first example of first processing in the embodiment of the present invention and showing the distal end portion the insertion portion and the observation object;

FIG. 8B is an explanatory diagram showing the first example of the first processing in the embodiment of the present invention and showing a three-dimensional image of the observation object;

FIG. 8C is an explanatory diagram showing the first example of the first processing in the embodiment of the present invention and showing a three-dimensional image after the first processing is performed;

FIG. 9A is an explanatory diagram showing a second example of the first processing in the embodiment of the present invention and showing the distal end portion of the insertion portion and the observation object;

FIG. 9B is an explanatory diagram showing the second example of the first processing in the embodiment of the present invention and showing a three-dimensional image of the observation object;

FIG. 9C is an explanatory diagram showing the second example of the first processing in the embodiment of the present invention and showing a three-dimensional image after the first processing is performed;

FIG. 10 is an explanatory diagram showing a display position of each of a left-eye image and a right-eye image in the embodiment of the present invention;

FIG. 11A is an explanatory diagram showing a first example of second processing in the embodiment of the present invention and showing the distal end portion of the insertion portion and the observation object;

FIG. 11B is an explanatory diagram showing the first example of the second processing in the embodiment of the present invention and showing a three-dimensional image of the observation object;

FIG. 11C is an explanatory diagram showing the first example of the second processing in the embodiment of the present invention and showing a three-dimensional image after the second processing is performed;

FIG. 12A is an explanatory diagram showing a second example of the second processing in the embodiment of the present invention and showing the distal end portion of the insertion portion and the observation object;

FIG. 12B is an explanatory diagram showing the second example of the second processing in the embodiment of the present invention and showing a three-dimensional image of the observation object;

FIG. 12C is an explanatory diagram showing the second example of the second processing in the embodiment of the present invention and showing a three-dimensional image after the second processing is performed; and

FIG. 13 is an explanatory diagram for describing operation of a line-of-sight direction detecting unit in the embodiment of the present invention.

### DETAILED DESCRIPTION OF THE PREFERRED EMBODIMENT

An embodiment of the present invention will be described below with reference to the drawings.

### Configuration of Endoscope System

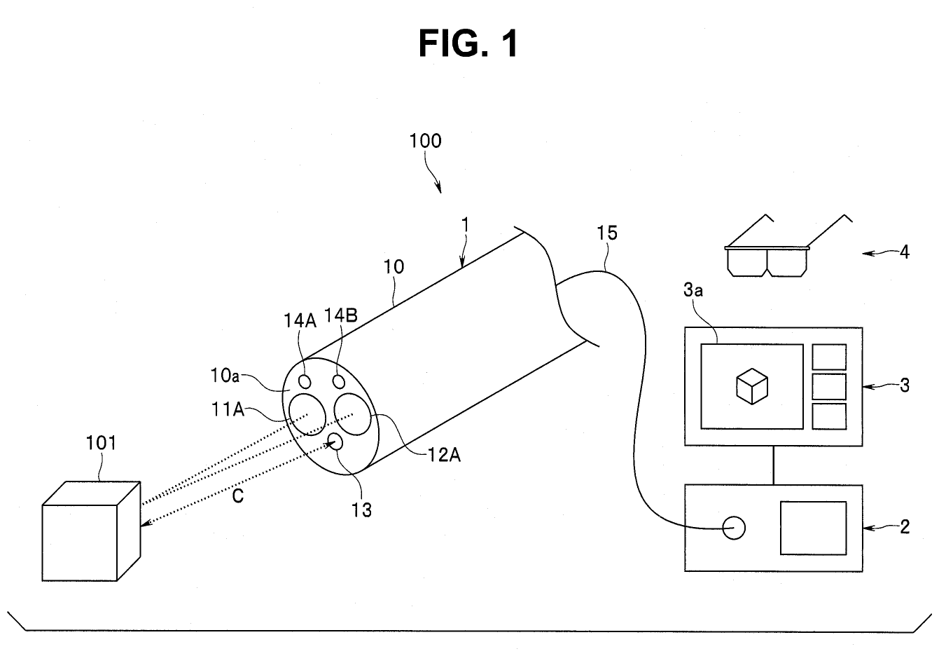

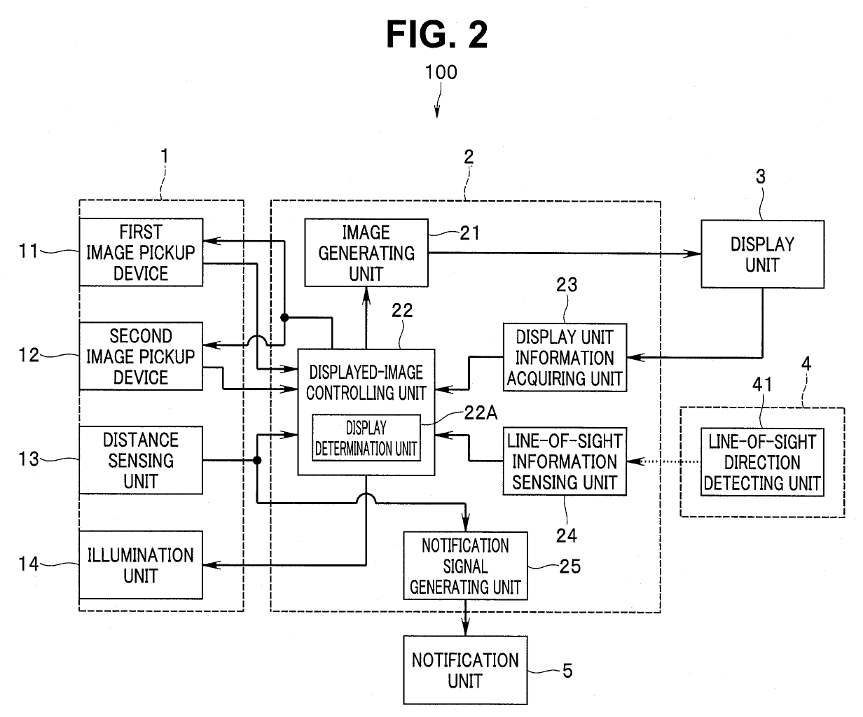

First, a schematic configuration of an endoscope system according to an embodiment of the present invention will be described. An endoscope system 100 according to the present embodiment is a three-dimensional endoscope system including a three-dimensional endoscope. FIG. 1 is an explanatory diagram showing a schematic configuration of the endoscope system 100. FIG. 2 is a functional block diagram showing a configuration of the endoscope system 100.

The endoscope system 100 includes a three-dimensional endoscope (hereinafter simply referred to as an endoscope) 1, a main-body device 2 having a function of a 3D video processor, a display unit 3 having a function of a 3D monitor, and 3D observation glasses 4 worn for seeing the display unit 3 to perceive a stereoscopic image. The endoscope 1 and the display unit 3 are connected to the main-body device 2. The 3D observation glasses 4 are configured to be able to communicate with the main-body device 2 through wired or wireless communication.

The endoscope 1 includes an insertion portion 10 inserted into a subject, an operation portion (not shown) connected to a proximal end of the insertion portion 10, and a universal cord 15 extending out from the operation portion. The endoscope 1 is connected to the main-body device 2 via the universal cord 15. The endoscope 1 may be constituted as a hard three-dimensional endoscope in which the insertion portion 10 has a hard tube portion, or may be constituted as a soft three-dimensional endoscope in which the insertion portion has a flexible tube portion.

The endoscope 1 also includes an image pickup optical system including a first image pickup device 11 and a second image pickup device 12 that pick up images of an object in a subject and an illumination optical system including an illumination unit 14. The image pickup optical system is provided at a distal end portion of the insertion portion 10. The image pickup optical system further includes two observation windows 11A and 12A provided on a distal end surface 10a of the insertion portion 10. The observation windows 11A and 12A constitute an end surface (hereinafter referred to as an objective surface) positioned at the distal end of the image pickup optical system. A light receiving surface of the first image pickup device 11 receives incident light from the object through the observation window 11A. A light receiving surface of the second image pickup device 12 receives incident light from the object through the observation window 12A. The first and second image pickup devices 11 and 12 are constituted by CCD or CMOS, for example.

The illumination optical system further includes two illumination windows 14A and 14B provided on the distal end surface 10a of the insertion portion 10. The illumination unit 14 emits illumination light for illuminating the object. The illumination light is emitted from the illumination windows 14A and 14B and irradiates the object. The illumination unit 14 may be provided at a position distanced from the distal end portion of the insertion portion 10. In this case, the illumination light emitted by the illumination unit 14 is transmitted to the illumination windows 14A and 14B by a lightguide provided in the endoscope 1. Alternatively, the illumination unit 14 may be constituted by a light-emitting element such as an LED provided at the distal end portion of the insertion portion 10.

The endoscope 1 further includes a distance sensing unit 13 that senses distance information that is information of a distance from the observation windows 11A and 12A, which constitute the objective surface, to a predetermined observation object 101 in the subject. In the present embodiment, the distance sensing unit 13 is provided on the distal end surface 10a of the insertion portion 10 in the same way as the observation windows 11A and 12A. The distance sensing unit 13 calculates the distance from the observation windows 11A and 12A to the observation object 101 based on a result of measuring a distance from the distal end surface 10a to the observation object 101 and a positional relationship between the observation windows 11A and 12A and the distance sensing unit 13. Hereinafter, it is assumed that a distance from the observation window 11A to the observation object 101 and a distance from the observation window 11A to the observation object 101 are equal to each other. The distance from the observation windows 11A and 12A to the observation object 101 is denoted by a symbol C. Note that, in FIG. 1, a distance from the distance sensing unit 13 to the observation object 101 is indicated by the symbol C, for convenience. The distance sensing unit 13 is constituted by a sensor that measures a distance to a measurement object by means of laser, infrared light, and ultrasound, for example.

The display unit 3 displays a 3D image generated from first and second picked-up images, which will be described later, as a displayed image. The 3D observation glasses 4 are glasses worn for seeing the 3D image displayed on the display unit 3 to observe the first picked-up image and the second picked-up image with respective, left and right eyes to perceive a stereoscopic image. The display unit 3 may be a polarized 3D monitor that displays the 3D image through different polarizing filters, or may be an active shutter 3D monitor that alternately displays the first picked-up image and the second picked-up image as the 3D image, for example. The 3D observation glasses 4 are polarized glasses if the display unit 3 is a polarized 3D monitor, and are shutter glasses if the display unit 3 is an active shutter 3D monitor.

The 3D observation glasses 4 include a line-of-sight direction detecting unit 41 that detects a direction of a line of sight of a wearer. A detection result of the line-of-sight direction detecting unit 41 is sent to the main-body device 2 through wired or wireless communication.

The endoscope 1 further includes a notification unit 5 connected to the main-body device 2. The notification unit 5 will be described later.

### Configuration of Main-Body Device

Next, a configuration of the main-body device 2 will be described with reference to FIG. 2. The main-body device 2 includes an image generating unit 21, a displayed-image controlling unit 22, a display unit information acquiring unit 23, a line-of-sight information sensing unit 24, and a notification signal generating unit 25.

The displayed-image controlling unit 22 performs, on the first picked-up image picked up by the first image pickup device 11 and the second picked-up image picked up by the second image pickup device 12, predetermined image processing and processing for outputting the first and second picked-up images as a 3D image, and outputs the processed first and second picked-up images to the image generating unit 21. As the processing for outputting the first and second picked-up images as a 3D image, processing of cutting out output regions for the 3D image, processing of controlling parameters required for displaying the 3D image, and the like are performed on the first picked-up image and the second picked-up image.

The image generating unit 21 generates a 3D image based on the first and second picked-up images outputted from the displayed-image controlling unit 22, and outputs the generated 3D image to the display unit 3. In the present embodiment, the image generating unit 21 is controlled by the displayed-image controlling unit 22 to perform predetermined image processing in generating the 3D image. The details of the image processing of the image generating unit 21 will be described later.

The display unit information acquiring unit 23 acquires display unit information that is information of a display region 3a of the display unit 3 connected to the main-body device 2, and is configured to be able to acquire the display unit information from the display unit 3. The display unit information includes, as the information of the display region 3a, information of a size of the display region 3a, that is, a dimension of the display region 3a in a vertical direction and a dimension of the display region 3a in a lateral direction, for example. The display unit information acquiring unit 23 outputs the acquired display unit information to the displayed-image controlling unit 22.

The line-of-sight information sensing unit 24 receives the detection result of the line-of-sight direction detecting unit 41 of the 3D observation glasses 4, and senses line-of-sight information that is information of movement of the direction of the line of sight based on the detection result of the line-of-sight direction detecting unit 41. The line-of-sight information sensing unit 24 outputs the sensed line-of-sight information to the displayed-image controlling unit 22.

The displayed-image controlling unit 22 can display a changed 3D image on the display unit 3 by controlling at least one of the endoscope 1, the image generating unit 21, and the display unit 3 and outputting the first and second picked-up images to the image generating unit 21 or providing the first and second picked-up images with control parameters for generating the 3D image. The displayed-image controlling unit 22 includes a display determination unit 22A that determines whether to display the 3D image on the display unit 3 based on the distance information sensed by the distance sensing unit 13. In the present embodiment, the displayed-image controlling unit 22 performs processing of changing the displayed image based on a determination result of the display determination unit 22A, the distance information sensed by the distance sensing unit 13, a content of the display unit information acquired by the display unit information acquiring unit 23, and a sensing result of the line-of-sight information sensing unit 24. The details of the processing of changing the displayed image will be described later.

The notification signal generating unit 25 generates a notification signal based on the distance information sensed by the distance sensing unit 13. For example, when the distance C from the observation windows 11A and 12A to the observation object 101 becomes a distance at which the observation object 101 is hard to recognize, the notification signal generating unit 25 generates a notification signal that notifies an observer to that effect. The notification signal generating unit 25 outputs the generated notification signal to the notification unit 5.

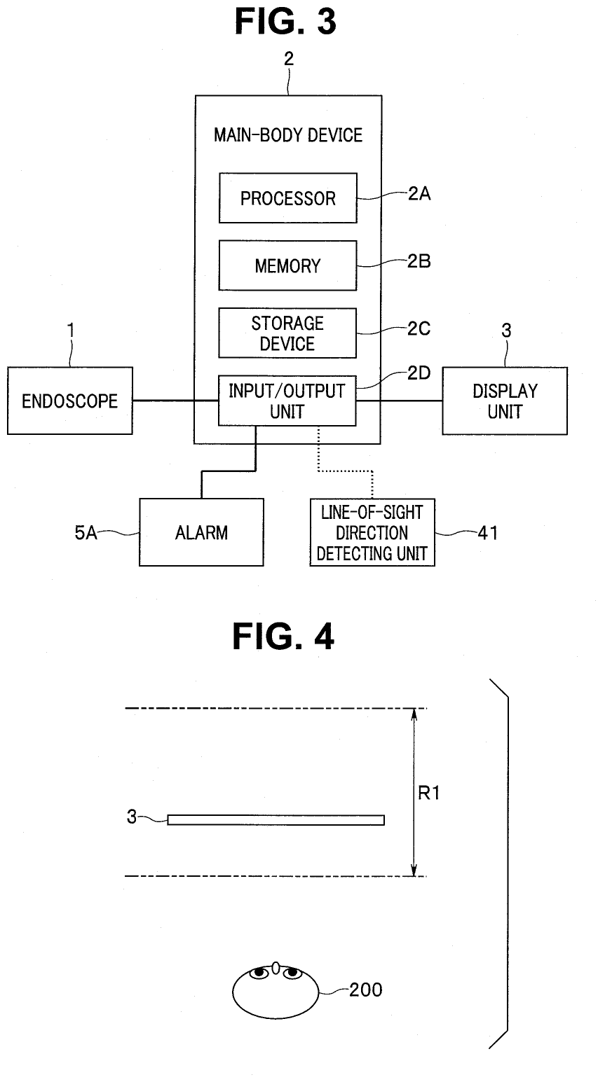

The notification unit 5 may be the display unit 3. In this case, the display unit 3 may display an alert that notifies the observer that the observation object 101 has become hard to recognize based on the notification signal. Note that, in FIG. 2, the display unit 3 and the notification unit 5 are shown as being separate, for convenience. Alternatively, the notification unit 5 may be an alarm 5A constituted by a speaker and the like. Note that the alarm 5A is shown in FIG. 3, which will be described later. The alarm 5A may notify the observer that the observation object 101 has become hard to recognize by means of voice or alarm sound based on the notification signal, for example.

Here, a hardware configuration of the main-body device 2 will be described with reference to FIG. 3. FIG. 3 is explanatory diagram showing an example of the hardware configuration of the main-body device 2. In the example shown in FIG. 3, the main-body device 2 includes a processor 2A, a memory 2B, a storage device 2C, and an input/output device 2D.

The processor 2A is used to perform at least part of functions of the image generating unit 21, the displayed-image controlling unit 22, the display unit information acquiring unit 23, the line-of-sight information sensing unit 24, and the notification signal generating unit 25. The processor 2A is constituted by an FPGA (field programmable gate array), for example. At least part of the image generating unit 21, the displayed-image controlling unit 22, the display unit information acquiring unit 23, the line-of-sight information sensing unit 24, and the notification signal generating unit 25 may be constituted as a circuit block in the FPGA.

The memory 2B is constituted by a rewritable volatile storage element such as a RAM. The storage device 2C is constituted by a rewritable non-volatile storage device such as a flash memory or a magnetic disk device. The input/output device 2D is used to send and receive signals between the main-body device 2 and an external device through wired or wireless communication.

Note that the processor 2A may be constituted by a central processing unit (hereinafter denoted as a CPU). In this case, at least part of the functions of the image generating unit 21, the displayed-image controlling unit 22, the display unit information acquiring unit 23, the line-of-sight information sensing unit 24, and the notification signal generating unit 25 may be implemented by the CPU reading out a program from the storage device 2C or another storage device that is not shown and executing the program.

The hardware configuration of the main-body device 2 is not limited to the example shown in FIG. 3. For example, each of the image generating unit 21, the displayed-image controlling unit 22, the display unit information acquiring unit 23, the line-of-sight information sensing unit 24, and the notification signal generating unit 25 may be constituted as a separate electronic circuit.

### Processing of Changing Displayed Image

Next, the processing of changing the displayed image performed by the displayed-image controlling unit 22 will be described in detail with reference to FIGS. 1 and 2. Processing included in the processing of changing the displayed image and other than processing performed based on the line-of-sight information will be described here. In the present embodiment, the displayed-image controlling unit 22 can selectively perform first processing, second processing, third processing, fourth processing, fifth processing, sixth processing, and seventh processing as the processing of changing the displayed image. The displayed-image controlling unit 22 performs these pieces of processing based on the determination result of the display determination unit 22A.

First, operation of the display determination unit 22A will be described. As described above, the display determination unit 22A determines whether to display the 3D image on the display unit 3 based on the distance information sensed by the distance sensing unit 13. More specifically, for example, when the distance C from the observation windows 11A and 12A to the observation object 101 is within a predetermined range, the display determination unit 22A determines that the 3D image is displayed on the display unit 3. On the other hand, when the distance C is out of the predetermined range, the display determination unit 22A determines that the 3D image is not displayed on the display unit 3. The predetermined range mentioned above is hereinafter referred to as a display determination range.

The display determination range is stored in advance in the storage device 2C shown in FIG. 3, a storage device that is not shown, or the like. The display determination unit 22A is configured to be able to read out the display determination range stored in the storage device 2C or the like. Note that first to third ranges, which will be described later, are also stored in advance in the storage device 2C or the like in the same way as the display determination range. The displayed-image controlling unit 22 is configured to be able to read out the first to third ranges stored in the storage device 2C or the like.

The display determination range is defined such that the distance C is within the display determination range when the distance C is a distance at which the observation object 101 can be comfortably observed or is a distance at which the observation object 101 is hard to recognize but the difficulty in recognizing the stereoscopic image can be resolved by performing the processing of changing the displayed image. In other words, when the observation object 101 can be comfortably observed or the difficulty in recognizing the stereoscopic image can be resolved by performing the processing of changing the displayed image, the display determination unit 22A determines that the 3D image is displayed on the display unit 3. On the other hand, when the difficulty in recognizing the stereoscopic image cannot be resolved even by performing the processing of changing the displayed image, the display determination unit 22A determines that the 3D image is not displayed on the display unit 3.

The first to sixth processing are performed when the display determination unit 22A determines that the 3D image is displayed on the display unit 3. The seventh processing is performed when the display determination unit 22A determines that the 3D image is not displayed on the display unit 3. Note that the first to sixth processing may be performed regardless of the determination of the display determination unit 22A.

Note that “the observation object 101 can be comfortably observed” more specifically means that a three-dimensional image of the observation object 101 can be observed without causing unnatural feeling and eyestrain, for example.

The distance C from the observation windows 11A and 12A to the observation object 101 at which the observation object 101 can be comfortably observed has a correspondence with a position at which the three-dimensional image of the observation object 101 is perceived. FIG. 4 schematically shows a range R1 in which a three-dimensional image of a stereoscopic image can be comfortably observed when an observer observes a 3D monitor. In FIG. 4, a reference numeral 200 indicates the observer. In FIG. 4, the display unit 3 is shown as the 3D monitor. As shown in FIG. 4, the range R1 in which the three-dimensional image of the stereoscopic image can be comfortably observed is a range from a predetermined first position closer to the observer 200 than the display unit 3 to a predetermined second position farther from the observer 200 than the display unit 3.

The position at which the three-dimensional image of the observation object 101 is perceived changes depending on the distance C from the observation windows 11A and 12A to the observation object 101. The distance C from the observation windows 11A and 12A to the observation object 101 is hereinafter denoted as a distance C between the observation object and the objective or simply as a distance C. As the distance C relatively decreases, the position at which the three-dimensional image of the observation object 101 is perceived becomes closer to the observer 200. As the distance C relatively increases, the position at which the three-dimensional image of the observation object 101 is perceived becomes farther from the observer 200. The distance C at which the observation object 101 can be comfortably observed is a distance such that the position at which the three-dimensional image of the observation object 101 is perceived is within the range R1 shown in FIG. 4.

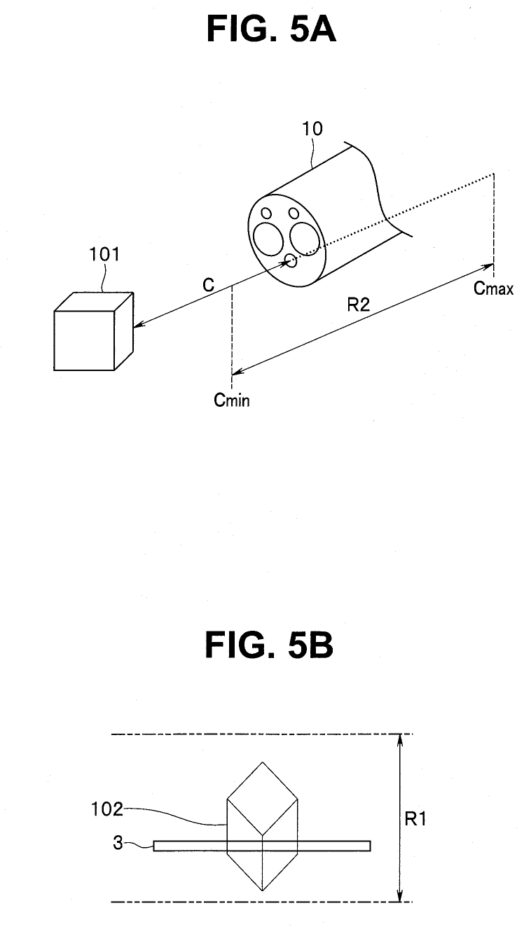

FIGS. 5A and 5B schematically show a range R2 of the distance C between the observation object and the objective in which a three-dimensional image of the stereoscopic image can be comfortably observed. FIG. 5A shows the distal end portion of the insertion portion 10 and the observation object 101, and FIG. 5B shows a three-dimensional image 102 of the observation object 101 when the distal end portion of the insertion portion 10 and the observation object 101 are in a positional relationship shown in FIG. 5A. In FIG. 5A, a symbol Cmin indicates a minimum value of the distance C at which the observation object 101 can be comfortably observed, and a symbol Cmax indicates a maximum value of the distance C at which the observation object 101 can be comfortably observed. As shown in FIG. 5B, when the distance C is within the range R2, the position at which the three-dimensional image 102 is perceived is within the range R1.

Next, the first processing will be described. In the first processing, the displayed-image controlling unit 22 controls the first and second picked-up images acquired by the first image pickup device 11 and the second image pickup device 12 of the endoscope 1. The displayed-image controlling unit 22 controls the first picked-up image so as to change a position of a first output region included in an entire image pickup region of the first image pickup device 11 and in which an image pickup signal for the first picked-up image is outputted based on the distance information sensed by the distance sensing unit 13. The displayed-image controlling unit 22 also controls the second picked-up image so as to change a position of a second output region included in an entire image pickup region of the second image pickup device 12 and in which an image pickup signal for the second picked-up image is outputted based on the distance information.

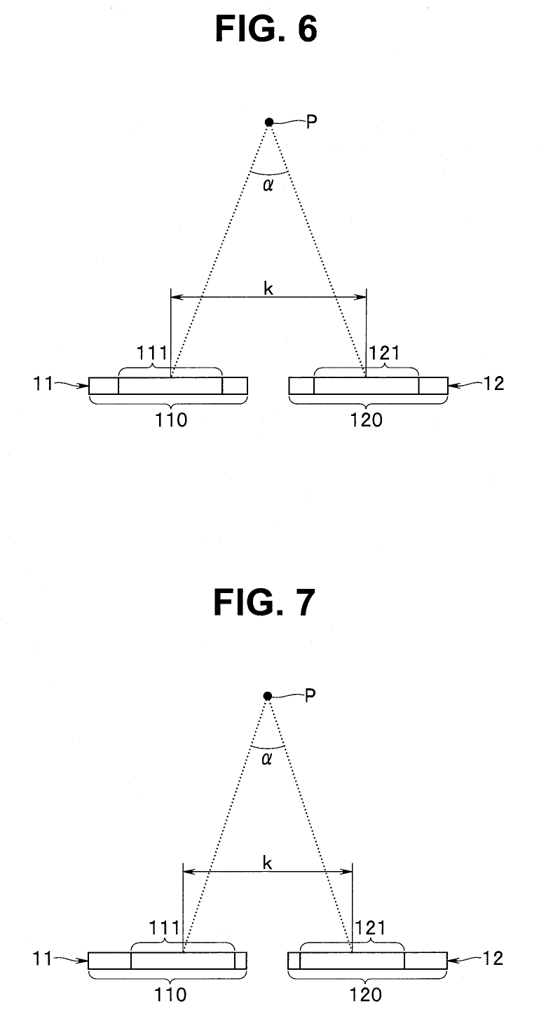

The details of the first processing will be specifically described below with reference to FIGS. 6 to 9C. FIG. 6 is an explanatory diagram showing image pickup regions and output regions of the image pickup devices 11 and 12. Note that, in FIG. 6, the image pickup regions of the first and second image pickup devices 11 and 12 are schematically indicated by respective, laterally long rectangles. Lengths of the rectangles in a left-right direction in FIG. 6 indicate dimensions of the image pickup regions of the first and second image pickup devices 11 and 12 in a direction parallel to a direction in which the first and second image pickup devices 11 and 12 are arranged.

In FIG. 6, a reference numeral 110 indicates the entire image pickup region of the first image pickup device 11, and a reference numeral 111 indicates the first output region in which the image pickup signal for the first picked-up image is outputted. A reference numeral 120 indicates the entire image pickup region of the second image pickup device 12, and a reference numeral 121 indicates the second output region in which the image pickup signal for the second picked-up image is outputted. As shown in FIG. 6, the first output region 111 is smaller than the entire image pickup region 110 of the first image pickup device 11, and the second output region 121 is smaller than the entire image pickup region 120 of the second image pickup device 12.

In FIG. 6, a point given with a symbol P indicates a point at which an optical axis (hereinafter referred to as a first optical axis) of an optical system including the first image pickup device 11 and the observation window 11A (see FIG. 1) and an optical axis (hereinafter referred to as a second optical axis) of an optical system including the second image pickup device 12 and the observation window 12A (see FIG. 1) intersect. An angle formed by the above-mentioned two optical axes, namely, an inward angle is hereinafter denoted by a symbol α. The inward angle α is a parameter having a correspondence with a size of the three-dimensional image of the stereoscopic image in a depth direction. If the inward angle α is larger than a convergence angle, which is determined by a pupil distance, which is an interval between left and right eyes of a person, and a distance to the 3D monitor, three-dimensional appearance is emphasized. If the inward angle α is less than or equal to the convergence angle, the three-dimensional appearance is weakened. In other words, as the inward angle α decreases, the size of the three-dimensional image in the depth direction decreases, and the three-dimensional appearance is weakened. On the other hand, as the inward angle α increases, the size of the three-dimensional image in the depth direction increases, and the three-dimensional appearance is enhanced. An interval between a center of the first output region 111 and a center of the second output region 121 is denoted by a symbol k. The interval k is a distance between the first and second optical axes.

FIG. 6 shows a situation in which the observation object 101 (see FIG. 1) is at the point P and the distance C from the observation windows 11A and 12A to the observation object 101 is within a predetermined first range. Note that the first range is defined such that the distance C is within the first range when the distance C is a distance at which the observation object 101 can be comfortably observed, for example. More specifically, the range R2 shown in FIG. 5B is the first range. FIG. 6 shows a situation in which the observation object 101 can be comfortably observed.

In the present embodiment, when the distance C is out of the first range, that is, is a distance at which the observation object 101 is hard to recognize, the displayed-image controlling unit 22 controls the first and second picked-up images such that the interval k between the center of the first output region 111 and the center of the second output region 121 decreases as compared to when the distance C is within the first range.

FIG. 7 shows a situation after positions of the output regions 111 and 121 are changed from the situation shown in FIG. 6. In the situation shown in FIG. 7, the interval k between the center of the first output region 111 and the center of the second output region 121 is smaller than in the situation shown in FIG. 6. In the situation shown in FIG. 7, the inward angle α is smaller than in the situation shown in FIG. 6. As a result, the size of the three-dimensional image of the stereoscopic image in the depth direction is decreased, and the three-dimensional appearance is weakened.

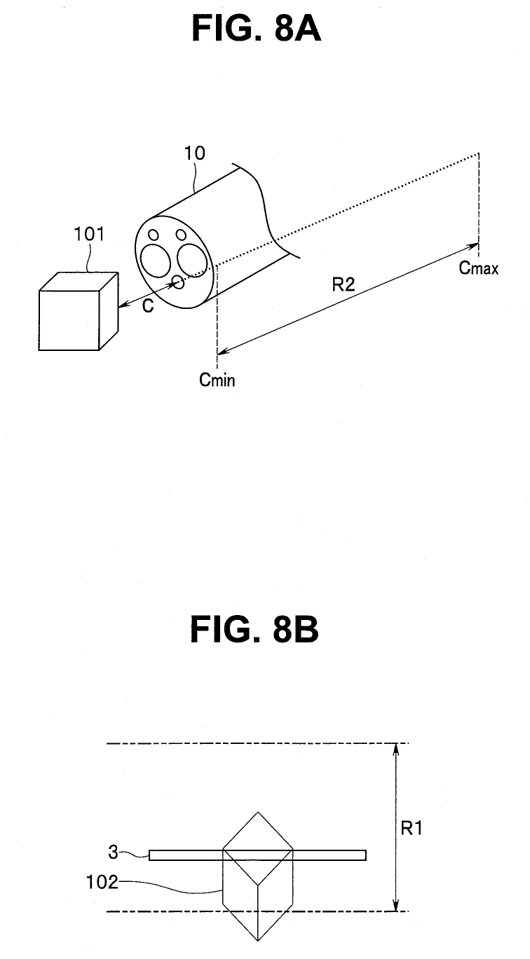

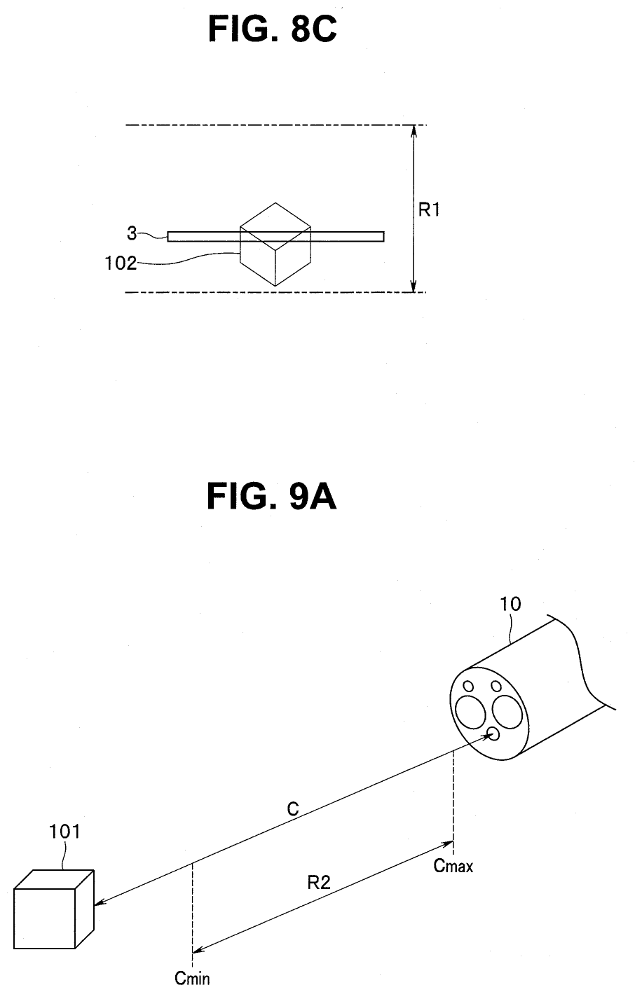

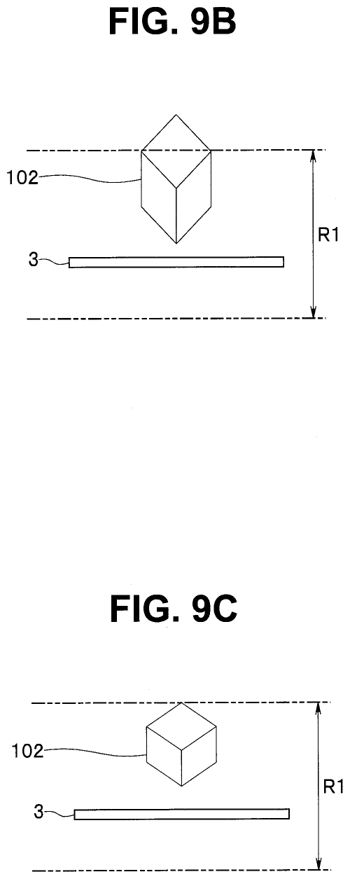

FIGS. 8A to 8C show a first example of the first processing. FIGS. 9A to 9C show a second example of the first processing. The first example is an example in which the distance C is smaller than the minimum value Cmin of the distance C at which the observation object 101 can be comfortably observed. The second example is an example in which the distance C is larger than the maximum value Cmax of the distance C at which the observation object 101 can be comfortably observed. FIGS. 8A and 9A show the distal end portion of the insertion portion 10 and the observation object 101, and FIGS. 8B and 9B show three-dimensional images 102 of the observation object 101 when the distal end portion of the insertion portion 10 and the observation object 101 are in positional relationships shown in FIGS. 8A and 9A. In the examples shown in FIGS. 8B and 9B, a part of the three-dimensional image 102 protrudes from the range R1 in which the three-dimensional image of the stereoscopic image can be comfortably observed.

FIGS. 8C and 9C show three-dimensional images 102 after the first processing is performed. In other words, FIGS. 8C and 9C show three-dimensional images 102 when the interval k is decreased as shown in FIG. 7. Note that FIGS. 8B and 9B show three-dimensional images 102 before the interval k is decreased as shown in FIG. 6. As shown in FIGS. 8A, 8C, 9B, and 9C, when the interval k is decreased to decrease the inward angle α, the size of the three-dimensional image 102 in the depth direction is decreased. As a result, the entire three-dimensional image 102 is included in the range R1 in which the three-dimensional image of the stereoscopic image can be comfortably observed.

Note that when decreasing the interval k between the center of the first output region 111 and the center of the second output region 121, the displayed-image controlling unit 22 may change the interval k in a stepwise or continuous manner according to the distance C.

The above has described the first processing for when the distance C from the observation windows 11A and 12A to the observation object 101 changes from being within the first range to being out of the first range. Conversely, when the distance C changes from being out of the first range to being within the first range, the displayed-image controlling unit 22 changes the interval k between the center of the first output region 111 and the center of the second output region 121 from the interval shown in FIG. 7 to the interval shown in FIG. 6.

Next, the second processing will be described. In the second processing, the image generating unit 21 is controlled by the displayed-image controlling unit 22. The displayed-image controlling unit 22 controls the image generating unit 21 so as to change a display position of each of a left-eye image and a right-eye image of the 3D image on the display unit 3 to change a position of the three-dimensional image 102 of the observation object 101 in the depth direction based on the distance information sensed by the distance sensing unit 13.

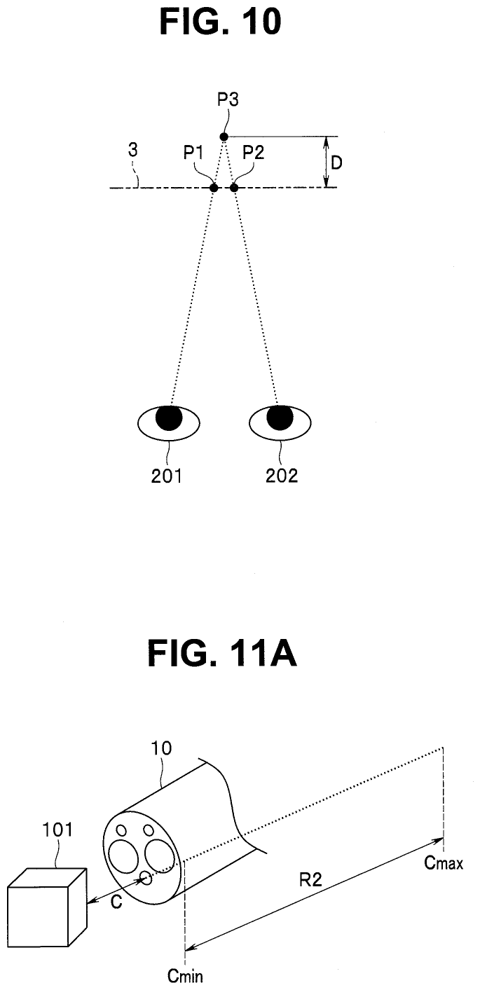

The details of the second processing will be specifically described below with reference to FIGS. 10 to 12C. FIG. 10 is an explanatory diagram showing the display position of each of the left-eye image and the right-eye image. In FIG. 10, a long dashed double-short dashed line given with a reference numeral 3 indicates a position of the display unit 3. A point given with a reference numeral P1 indicates a position of the observation object 101 in the left-eye image (see FIG. 1), and a point given with a reference numeral P2 indicates a position of the observation object 101 in the right-eye image. When the observation object 101 in the left-eye image (the point P1) is seen with a left eye 201 and the observation object 101 in the right-eye image (the point P2) is seen with a right eye 202, the three-dimensional image 102 of the observation object 101 is perceived as being positioned at a point given with a reference numeral P3. FIG. 10 shows an example in which the point P3 at which the observation object 101 is perceived is positioned deeper than the display unit 3.

In the present embodiment, when the distance C from the observation windows 11A and 12A to the observation object 101 is out of a predetermined second range, the displayed-image controlling unit 22 changes the display position of each of the left-eye image and the right-eye image on the display unit 3 such that a distance between the point P1 at which the observation object 101 is positioned in the left-eye image and the point P2 at which the observation object 101 is positioned in the right-eye image decreases as compared to when the distance C is within the second range. In this manner, a distance D from the display unit 3 to the point P3 at which the three-dimensional image 102 of the observation object 101 is perceived, that is, a stereoscopic depth of the three-dimensional image 102 of the observation object 101 is decreased.

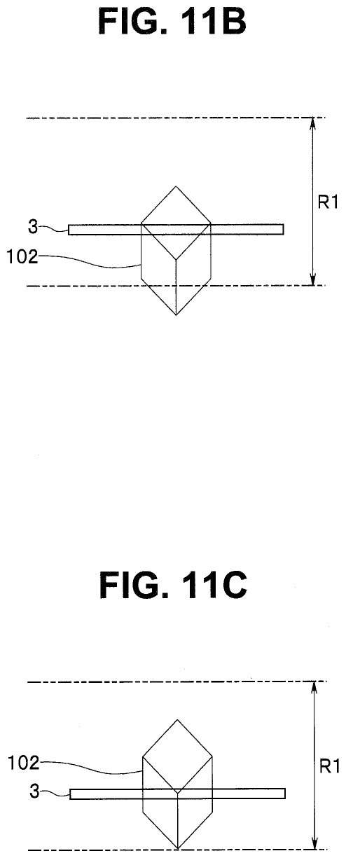

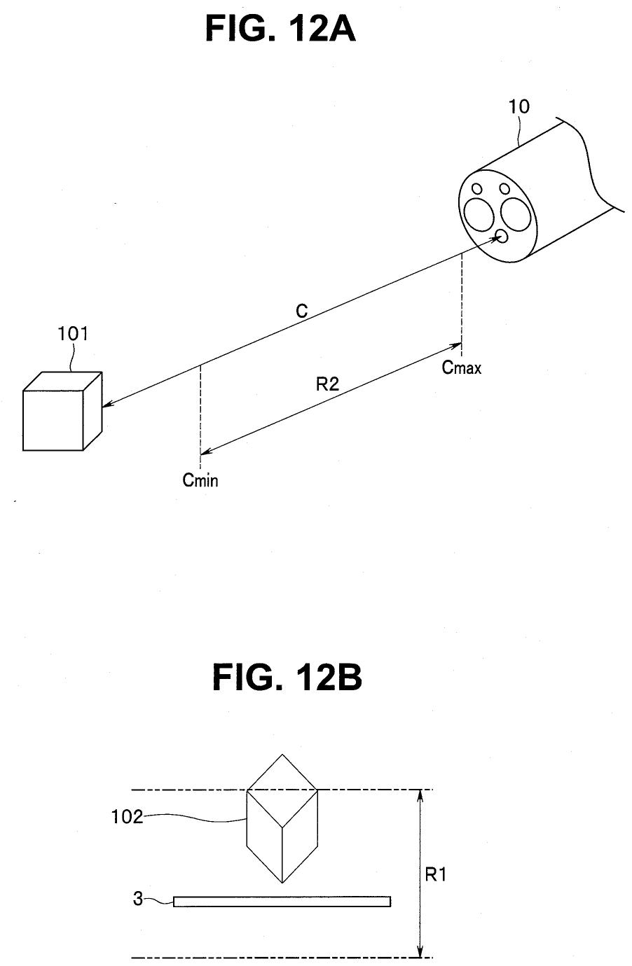

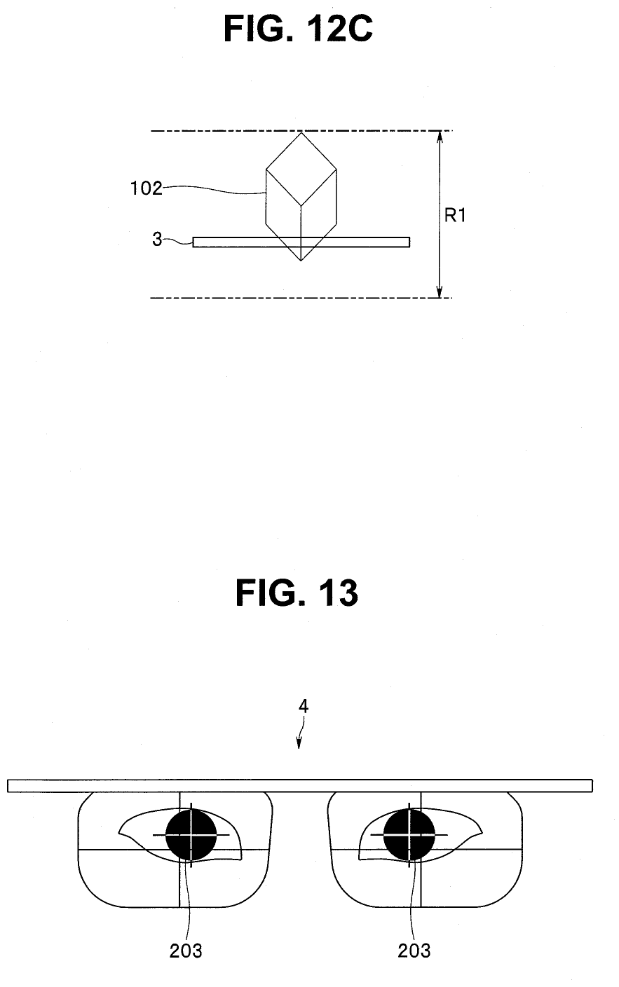

FIGS. 11A to 11C show a first example of the second processing. FIGS. 12A to 12C show a second example of the second processing. The first example is an example in which the distance C is smaller than the minimum value Cmin of the distance C at which the observation object 101 can be comfortably observed. The second example is an example in which the distance C is larger than the maximum value Cmax of the distance C at which the observation object 101 can be comfortably observed. FIGS. 11A and 12A show the distal end portion of the insertion portion 10 and the observation object 101, and FIGS. 11B and 12B show three-dimensional images 102 of the observation object 101 when the distal end portion of the insertion portion 10 and the observation object 101 are in positional relationships shown in FIGS. 11A and 12A. In the examples shown in FIGS. 11B and 12B, a part of the three-dimensional image 102 protrudes from the range R1 in which the three-dimensional image of the stereoscopic image can be comfortably observed.

FIGS. 11C and 12C show three-dimensional images 102 after the second processing is performed. In other words, FIGS. 11C and 12C show three-dimensional images 102 when the distance between the point P1 and the point P2 shown in FIG. 10 is decreased. Note that FIGS. 11B and 12B show three-dimensional images 102 before the distance between the point P1 and the point P2 is decreased. As shown in FIGS. 11B, 11C, 12B, and 12C, when the distance between the point P1 and the point P2 is decreased, the stereoscopic depth of the three-dimensional image 102 is decreased, and the position of the three-dimensional image 102 in the depth direction changes such that the entire three-dimensional image 102 is included in the range R1 in which the three-dimensional image of the stereoscopic image can be comfortably observed. Note that, in the second processing, the size of the three-dimensional image 102 in the depth direction does not change, unlike the first processing.

Note that the second range may be defined in the same way as the first range, for example. In this case, when the distance C is a distance at which the observation object 101 is hard to recognize, the first processing and the second processing are performed at the same time.

Alternatively, the second range may be defined such that the distance C is within the second range when there is a distance at which the observation object 101 is hard to recognize but the observation object 101 can be comfortably observed by performing the first processing. If the second range is defined in this manner, when the distance C is out of the second range, that is, when the observation object 101 cannot be comfortably observed even by performing the first processing, the first processing and the second processing are performed at the same time. Note that when there is a distance at which the observation object 101 is hard to recognize but the observation object 101 can be comfortably observed by performing the first processing, only the first processing is performed and the second processing is not performed.

When decreasing the distance between the point P1 and the point P2, the displayed-image controlling unit 22 may change the distance between the point P1 and the point P2 in a stepwise or continuous manner according to the distance C.

Next, the third processing will be described. In the third processing, the illumination unit 14 of the endoscope 1 is controlled by the displayed-image controlling unit 22. The displayed-image controlling unit 22 controls the illumination unit 14 so as to change a light quantity of the illumination light based on the distance information sensed by the distance sensing unit 13.

The third processing is performed when the distance C from the observation windows 11A and 12A to the observation object 101 is a distance at which the observation object 101 cannot be comfortably observed even by performing the first processing, for example. When the distance C is relatively small, the displayed-image controlling unit 22 controls the illumination unit 14 such that the light quantity of the illumination light increases to cause halation. When the distance C is relatively large, the displayed-image controlling unit 22 controls the illumination unit 14 such that the light quantity of the illumination light decreases to darken the stereoscopic image. Note that when increasing or decreasing the light quantity of the illumination light as described above, the displayed-image controlling unit 22 may change the light quantity of illumination light in a stepwise or continuous manner according to the distance C.

Next, the fourth processing will be described. In the fourth processing, the image generating unit 21 is controlled by the displayed-image controlling unit 22. The displayed-image controlling unit 22 controls the image generating unit 21 so as to perform blurring processing on the 3D image based on the distance information sensed by the distance sensing unit 13. The fourth processing is performed when the distance C from the observation windows 11A and 12A to the observation object 101 is a distance at which the observation object 101 cannot be comfortably observed even by performing the first processing, for example. Note that when performing the blurring processing, the displayed-image controlling unit 22 may change a degree of blurring in a stepwise or continuous manner according to the distance C.

Next, the fifth processing will be described. In the fifth processing, the image generating unit 21 is controlled by the displayed-image controlling unit 22. The displayed-image controlling unit 22 controls the image generating unit 21 so as to change an area of each of the left-eye image and the right-eye image of the 3D image displayed on the display unit 3 based on the display unit information acquired by the display unit information acquiring unit 23.

As the display region 3a of the display unit 3 becomes relatively larger, that is, the dimension of the display region 3a in the vertical direction and the dimension of the display region 3a in the lateral direction relatively increase, a position of a three-dimensional image near an outer edge of a perceived range of the stereoscopic image becomes farther from the display unit 3. The fifth processing is performed when the display region 3a of the display unit 3 is larger than a predetermined threshold, for example. In this case, the displayed-image controlling unit 22 controls the image generating unit 21 so as to delete a portion near the outer edge of each of the left-eye image and the right-eye image displayed on the display unit 3 to decrease the area of each of the left-eye image and the right-eye image.

Next, the sixth processing will be described. In the sixth processing, the image generating unit 21 is controlled by the displayed-image controlling unit 22. The displayed-image controlling unit 22 controls the image generating unit 21 so as to change the display position of each of the left-eye image and the right-eye image of the 3D image on the display unit 3 based on the distance information sensed by the distance sensing unit 13 and the display unit information acquired by the display unit information acquiring unit 23. More specifically, for example, when the display region 3a of the display unit 3 is larger than a predetermined threshold and the distance C from the observation windows 11A and 12A to the observation object 101 is out of a predetermined third range, the displayed-image controlling unit 22 changes the display position of each of the left-eye image and the right-eye image on the display unit 3 such that the distance between the point P1 at which the observation object 101 is positioned in the left-eye image and the point P2 at which the observation object 101 is positioned in the right-eye image (see FIG. 10) decreases as compared to when the distance C is within the third range.

Note that the third range may be defined in the same way as the second range, for example. When decreasing the distance between the point P1 and the point P2, the displayed-image controlling unit 22 may change the distance between the point P1 and the point P2 in a stepwise or continuous manner according to the distance C.

Next, the seventh processing will be described. As described above, the seventh processing is performed when the display determination unit 22A determines that the 3D image is not displayed on the display unit 3. In the seventh processing, the image generating unit 21 is controlled by the displayed-image controlling unit 22. The displayed-image controlling unit 22 controls the image generating unit 21 so as to generate a single 2D image based on the first and second picked-up images. For example, the image generating unit 21 may use one of the first and second picked-up images as the 2D image generated by the image generating unit 21. The display unit 3 displays the 2D image generated by the image generating unit 21.

Note that the displayed-image controlling unit 22 may be configured to be able to perform all of the first to seventh processing, or may be configured to be able to perform the first processing and at least one of the second to seventh processing.

### Processing Performed Based on Line-of-Sight Information

Next, the processing performed based on the line-of-sight information included in the processing of changing the displayed image will be described. First, operation of the line-of-sight direction detecting unit 41 of the 3D observation glasses 4 and operation of the line-of-sight information sensing unit 24 will be described with reference to FIGS. 2 and 13. FIG. 13 is an explanatory diagram for describing the operation of the line-of-sight direction detecting unit 41. For example, the line-of-sight direction detecting unit 41 is constituted by a sensor that is not shown, such as a camera that detects positions of pupils 203, and detects the direction of the line of sight of the wearer by detecting the positions of the pupils 203. Based on a detection result of the line-of-sight direction detecting unit 41, that is, a detection result of the direction of the line of sight of the wearer, the line-of-sight information sensing unit 24 senses line-of-sight information that is information of movement of the direction of the line of sight.

Next, the processing performed based on the line-of-sight information will be described. The displayed-image controlling unit 22 performs the processing of changing the displayed image based on the line-of-sight information sensed by the line-of-sight information sensing unit 24. In the present embodiment, when an amount of movement of the direction of the line of sight within a predetermined period of time is greater than or equal to a predetermined threshold, the displayed-image controlling unit 22 controls the endoscope 1 and the image generating unit 21 to perform at least the first processing among the foregoing first to seventh processings. Note that the displayed-image controlling unit 22 may perform the above-mentioned processing regardless of the distance information sensed by the distance sensing unit 13 when the amount of movement of the direction of the line of sight within the predetermined period of time is greater than or equal to the predetermined threshold. Alternatively, the displayed-image controlling unit 22 may perform the above-mentioned processing when the amount of movement of the direction of the line of sight within the predetermined period of time is greater than or equal to the predetermined threshold and the distance C from the observation windows 11A and 12A to the observation object 101 is out of a predetermined range. The above-mentioned predetermined range may be a range that is narrower than the foregoing first range, for example.

### Operations and Effects

Next, operations and effects of the endoscope system 100 according to the present embodiment will be described. In the present embodiment, the displayed-image controlling unit 22 can perform processing of controlling the first picked-up image so as to change the position of the first output region 111 and controlling the second picked-up image so as to change the position of the second output region 121 based on the distance information that is information of the distance C from the observation windows 11A and 12A to the observation object 101 (the first processing). As described above, as the interval k between the center of the first output region 111 and the center of the second output region 121 decreases, the inward angle α decreases, and as the inward angle α decreases, the size of the three-dimensional image in the depth direction decreases. Thus, the three-dimensional appearance is weakened. Therefore, according to the present embodiment, when there is a distance at which the observation object 101 is hard to recognize, by controlling the first and second picked-up images such that the interval k decreases, the three-dimensional appearance of the three-dimensional image of the observation object 101 is weakened, and the difficulty in recognizing the observation object 101 can be resolved. As a result, according to the present embodiment, the difficulty in recognizing the stereoscopic image can be resolved.

A value of the interval k when the distance C from the observation windows 11A and 12A to the observation object 101 is within the foregoing first range is referred to as a first value, and a value of the interval k when the distance C is out of the first range, which value is different from the first value, is referred to as a second value. In the present embodiment, the displayed-image controlling unit 22 controls the first and second picked-up images such that the interval k is at the first value when the distance C is within the first range, and controls the first and second picked-up images such that the interval k is at the second value when the distance C is out of the first range. In the present embodiment, in particular, the first range is defined such that the distance C is within the first range when the distance C is a distance at which the observation object 101 can be comfortably observed, and the second value is smaller than the first value. Thus, according to the present embodiment, when the distance C changes from being a distance at which the observation object 101 can be comfortably observed to being a distance at which the observation object 101 is hard to recognize, the three-dimensional appearance of the three-dimensional image of the observation object 101 can be weakened, and when the distance C changes back from being a distance at which the observation object 101 is hard to recognize to being a distance at which the observation object 101 can be comfortably observed, the three-dimensional appearance of the three-dimensional image of the observation object 101 can be restored.

Note that the second value may be a single value or a plurality of values as long as the above-mentioned requirement for the second value is met.

In the present embodiment, the interval k between the center of the first output region 111 and the center of the second output region 121 is electrically changed. Thus, according to the present embodiment, as compared to a case in which a mechanism for physically changing the interval k between the center of the first output region 111 and the center of the second output region 121 is provided, a structure of the distal end portion of the insertion portion 10 of the endoscope 1 can be simplified, and the distal end portion can be made smaller.

In the present embodiment, the displayed-image controlling unit 22 can perform processing of controlling the image generating unit 21 so as to change the display position of each of the left-eye image and the right-eye image of the 3D image on the display unit 3 (the second processing). Thus, according to the present embodiment, when there is a distance at which the observation object 101 is hard to recognize, by changing the display position of each of the left-eye image and the right-eye image on the display unit 3 such that the distance between the point P1 at which the observation object 101 is positioned in the left-eye image and the point P2 at which the observation object 101 is positioned in the right-eye image decreases, the stereoscopic depth of the three-dimensional image of the observation object 101 can be decreased. Thus, according to the present embodiment, the difficulty in recognizing the observation object 101 can be resolved, and as a result, the difficulty in recognizing the stereoscopic image can be resolved.

Note that the distance between the point P1 and the point P2 on the display unit 3 may be defined based on, for example, the distance C from the observation windows 11A and 12A to the observation object 101, the interval k between the center of the first output region 111 and the center of the second output region 121, the interval between the left eye 201 and the right eye 202 of the observer (the pupil distance), the distance from the display unit 3 to the observer, and the like, regardless of whether to perform the second processing.

In the present embodiment, the displayed-image controlling unit 22 can perform processing of controlling the illumination unit 14 so as to change the light quantity of the illumination light (the third processing). In the present embodiment, as described above, when the distance C from the observation windows 11A and 12A to the observation object 101 is a distance at which the observation object 101 cannot be comfortably observed even by performing the first processing, halation is caused or the stereoscopic image is darkened. In the present embodiment, by intentionally making the three-dimensional image of the observation object 101 harder to recognize in this manner, the difficulty in recognizing the stereoscopic image can be resolved.

In the present embodiment, the displayed-image controlling unit 22 can perform processing of controlling the image generating unit 21 so as to perform blurring processing on the 3D image (the fourth processing). According to the present embodiment, when the distance C from the observation windows 11A and 12A to the observation object 101 is a distance at which the observation object 101 cannot be comfortably observed even by performing the first processing, by intentionally making the three-dimensional image of the observation object 101 harder to recognize by performing the blurring processing, the difficulty in recognizing the stereoscopic image can be resolved.

In the present embodiment, the displayed-image controlling unit 22 can perform processing of controlling the image generating unit 21 so as to change the area of each of the left-eye image and the right-eye image of the 3D image displayed on the display unit 3 based on the display unit information acquired by the display unit information acquiring unit 23 (the fifth processing). In the present embodiment, as described above, by deleting a portion near the outer edge of each of the left-eye image and the right-eye image displayed on the display unit 3, a three-dimensional image near the outer edge of the perceived range of the stereoscopic image and at a portion far from the display unit 3 can be deleted. Thus, according to the present embodiment, the difficulty in recognizing the stereoscopic image can be resolved.

In the present embodiment, the displayed-image controlling unit 22 can perform processing of controlling the image generating unit 21 so as to change the display position of each of the left-eye image and the right-eye image of the 3D image on the display unit 3 based on the distance information and the display unit information (the sixth processing). In the present embodiment, as described above, when the display region 3a of the display unit 3 is larger than the predetermined threshold and the distance C from the observation windows 11A and 12A to the observation object 101 is out of the third range, by changing the display position of each of the left-eye image and the right-eye image on the display unit 3 such that the distance between the point P1 at which the observation object 101 is positioned in the left-eye image and the point P2 at which the observation object 101 is positioned in the right-eye image decreases, the stereoscopic depth of the three-dimensional image of the observation object 101 can be decreased. Thus, according to the present embodiment, the difficulty in recognizing the observation object 101 can be resolved, and as a result, the difficulty in recognizing the stereoscopic image can be resolved.

In the present embodiment, when the display determination unit 22A determines that the 3D image is not displayed on the display unit 3, the displayed-image controlling unit 22 can perform processing of controlling the image generating unit 21 so as to generate a single 2D image based on the first and second picked-up images (the seventh processing). Thus, according to the present embodiment, when the observation object 101 cannot be comfortably observed even by performing the processing of changing the displayed image, by displaying the 2D image, eyestrain or the like due to the difficulty in recognizing the stereoscopic image can be prevented.

When the observer attempts to observe a three-dimensional image that is at a position far from the display unit 3 and is hard to recognize, the image becomes out of focus, and the positions of the pupils and hence the direction of the line of sight fluctuate. Thus, a situation in which the direction of the line of sight fluctuates can be regarded as a situation in which the stereoscopic image is hard to recognize. In the present embodiment, when the amount of movement of the direction of the line of sight within the predetermined period of time is greater than or equal to the predetermined threshold, the displayed-image controlling unit 22 can control the endoscope 1 and the image generating unit 21 to perform at least the first processing among the foregoing first to seventh processing. Thus, according to the present embodiment, the difficulty in recognizing the stereoscopic image can be resolved.

When the display region 3a of the display unit 3 becomes relatively larger, a three-dimensional image of the observation object 101 positioned relatively far is perceived as being positioned farther from the observer, and a three-dimensional image of the observation object 101 positioned relatively close is perceived as being positioned closer to the observer. In this manner, as the display region 3a of the display unit 3 becomes larger, the range R1 (see FIG. 4) of distance at which the observation object 101 can be comfortably observed becomes smaller. As described above, the first range is defined such that the distance C from the observation windows 11A and 12A to the observation object 101 is within the first range when the distance C is a distance at which the observation object 101 can be comfortably observed, for example. The displayed-image controlling unit 22 may change the first range based on the display unit information. More specifically, when the display region 3a of the display unit 3 is relatively large, the displayed-image controlling unit 22 may reduce the first range. Thus, according to the present embodiment, the difficulty in recognizing the observation object 101 can be resolved, and as a result, the difficulty in recognizing the stereoscopic image can be resolved.

The present invention is not limited to the above-described embodiment, and various changes, modifications, and the like are possible without departing from the spirit of the present invention. For example, in the second processing, the fifth processing, and the sixth processing, the displayed-image controlling unit 22 may control the display unit 3, instead of controlling the image generating unit 21, to change the display position and area of the 3D image displayed on the display unit 3.

What is claimed is:

1. An endoscope system comprising:
an endoscope comprising a first image pickup device and a second image pickup device each configured to pick up an image of an object in a subject;
a monitor configured to display a 3D image as a displayed image;
a sensor configured to sense distance information that is information of a distance from an objective surface positioned at a distal end of an insertion portion of the endoscope to a predetermined observation object in the subject; and
a processor, wherein
the processor is configured to:
generate the 3D image based on a first picked-up image picked up by the first image pickup device and a second picked-up image picked up by the second image pickup device; and
change the displayed image by performing at least one of control of the endoscope, image processing for generating the 3D image, and control of the monitor, and
based on the distance information, the processor controls the first picked-up image so as to change a position of a first output region included in an entire image pickup region of the first image pickup device and in which an image pickup signal for the first picked-up image is outputted, controls the second picked-up image so as to change a position of a second output region included in an entire image pickup region of the second image pickup device and in which an image pickup signal for the second picked-up image is outputted, controls the first and second picked-up images such that an interval between a center of the first output region and a center of the second output region is a first value when the distance is within a predetermined range, and controls the first and second picked-up images such that the interval is a second value different from the first value when the distance is out of the predetermined range.

2. The endoscope system according to claim 1, wherein
the predetermined range is defined such that the distance is within the predetermined range when the distance is a distance at which the observation object can be comfortably observed, and
the second value is smaller than the first value.

3. The endoscope system according to claim 1, wherein
the processor performs the image processing or the control of the monitor so as to change a display position of each of a left-eye image and a right-eye image of the 3D image on the monitor based on the distance information.

4. The endoscope system according to claim 1, wherein
the processor is further configured to determine whether to display the 3D image on the monitor based on the distance information,
when the processor determines that the 3D image is displayed, the processor performs the image processing so as to generate the 3D image, and
when the processor determines that the 3D image is not displayed, the processor performs the image processing so as to generate a single 2D image based on the first and second picked-up images, and the monitor displays the 2D image as the displayed image.

5. The endoscope system according to claim 1, wherein
the endoscope further comprises an illuminator configured to emit illumination light for irradiating the object, and
the processor controls the illuminator so as to change a light quantity of the illumination light based on the distance information.

6. The endoscope system according to claim 1, wherein
the processor performs the image processing so as to perform blurring processing on the 3D image when the distance is out of a predetermined range.

7. The endoscope system according to claim 1, wherein
the processor is further configured to acquire display unit information that is information of a display region of the monitor, and
the processor performs at least one of the control of the endoscope, the image processing, and the control of the monitor to change the displayed image based on the distance information and the display unit information.

8. The endoscope system according to claim 7, wherein
the processor performs the image processing so as to change an area of each of a left-eye image and a right-eye image of the 3D image displayed on the monitor based on the display unit information.

9. The endoscope system according to claim 7, wherein
the processor performs the image processing or the control of the monitor so as to change a display position of each of a left-eye image and a right-eye image of the 3D image on the monitor based on the distance information and the display unit information.

10. The endoscope system according to claim 1, wherein
the processor is further configured to generate a notification signal based on the distance information.

11. The endoscope system according to claim 1,
further comprising 3D observation glasses worn for seeing the 3D image to perceive a stereoscopic image and configured to detect a direction of a line of sight of a wearer, wherein
the processor is further configured to sense line-of-sight information that is information of movement of the direction of the line of sight based on a detection result of the direction of the line of sight, and
the processor performs at least one of the control of the endoscope, the image processing, and the control of the monitor to change the displayed image when an amount of movement of the direction of the line of sight within a predetermined period of time is greater than or equal to a predetermined threshold.

12. An endoscopic image generating method for generating a 3D image based on first and second picked-up images respectively picked up by first and second image pickup devices of an endoscope,
the endoscopic image generating method comprising:
sensing distance information that is information of a distance from an objective surface positioned at a distal end of an insertion portion of the endoscope to a predetermined observation object in a subject; and
based on the distance information, controlling the first picked-up image so as to change a position of a first output region included in an entire image pickup region of the first image pickup device and in which an image pickup signal for the first picked-up image is outputted, controlling the second picked-up image so as to change a position of a second output region included in an entire image pickup region of the second image pickup device and in which an image pickup signal for the second picked-up image is outputted, controlling the first and second picked-up images such that an interval between a center of the first output region and a center of the second output region is a first value when the distance is within a predetermined range, and controlling the first and second picked-up images such that the interval is a second value different from the first value when the distance is out of the predetermined range.

13. A non-transitory computer-readable recording medium storing an endoscopic image processing program to be executed by a computer, wherein
the endoscopic image processing program causes an endoscopic image generating system for generating a 3D image based on first and second picked-up images respectively picked up by first and second image pickup devices of an endoscope to perform:
sensing distance information that is information of a distance from an objective surface positioned at a distal end of an insertion portion of the endoscope to a predetermined observation object in a subject; and
based on the distance information, controlling the first picked-up image so as to change a position of a first output region included in an entire image pickup region of the first image pickup device and in which an image pickup signal for the first picked-up image is outputted, controlling the second picked-up image so as to change a position of a second output region included in an entire image pickup region of the second image pickup device and in which an image pickup signal for the second picked-up image is outputted, controlling the first and second picked-up images such that an interval between a center of the first output region and a center of the second output region is a first value when the distance is within a predetermined range, and controlling the first and second picked-up images such that the interval is a second value different from the first value when the distance is out of the predetermined range.
